(19) United States
(12) Patent Application Publication
(10) Pub. No.: US 2005/0196518 A1
(43) Pub. Date: Sep. 8, 2005

(54) Method and system for making a coated medical device

(76) Inventor: Eric B. Stenzel, Tuam (IE)

Correspondence Address: JONES DAY;COUNSELORS AT LAW, 222 East 41st Street, New York, NY, 10017, US

(21) Appl. No.: 10/793,623

(22) Filed: Mar. 3, 2004

(51) Int. Cl. A61L 2/00; B05D 3/00

(52) U.S. Cl. 427/2.1; 118/20; 118/642

(57) ABSTRACT

Methods of making medical devices, such as stents, having a surface and a coating layer disposed on a portion of the surface are described herein. The coating is formed by applying a coating composition to a portion of the surface of the medical device and then at least partially drying the coating composition substantially simultaneously with the application of the coating composition. The process may be repeated until a desired amount of the coating composition is applied to the surface of the medical device. This method allows for a more efficient and effective method of applying a coating composition to a medical device such as a stent. Also disclosed is a system for making a coated medical device.

### FIELD OF THE INVENTION

This invention relates generally to coated medical devices. More particularly, the invention is directed to methods and systems for making medical devices having a coating on at least a portion of the surface of the medical device.

### BACKGROUND OF THE INVENTION

It has been common to treat a variety of medical conditions by introducing an insertable or implantable medical device having a coating for release of a biologically active material into a body lumen of a patient. For example, various types of drug-coated stents have been used for localized delivery of drugs to a body lumen. See, e.g., U.S. Pat. No. 6,099,562 to Ding et al.

Previously, such coated medical devices have been manufactured by shaping the body of a medical device such as a stent first by photo-etching, laser ablation, electron beam ablation, or any other means, and then coating a surface of the medical device with a coating composition which includes a solvent, a polymer dissolved in the solvent, and a therapeutic substance dispersed in the solvent. Conventionally, such coating compositions have been applied to a medical device by processes such as dipping, spraying, vapor deposition, plasma polymerization, and electrodeposition. After the coating composition has been applied to the medical device, the solvent was evaporated leaving the polymer/therapeutic agent coating.

Although these processes have been used to produce satisfactory coatings on medical devices, there are numerous potential drawbacks associated therewith. For example, many conventional processes require multiple coating steps or stages for the application of a second coating material, or to allow for complete drying between coating step which can increase production time. For example, the time needed to completely dry a coating composition between spray-coating passes is typically about 3.5 hours.

Also, it is often difficult to form coatings of uniform thicknesses, both on individual parts and on batches of parts, as conventional methods are prone to the formation of polymeric surface imperfections during the coating process. This is especially evident on stents, which generally include many struts with small interstitial spaces therebetween. When using dip-coating and spray-coating methods, there is the possibility of forming web-like defects or bridges by build-up of excess polymeric material between the stent struts. Dripping, bridging, and webbing occurs between the struts, particularly when the coating composition is sprayed too quickly. However, to reduce the possibility of dripping along the device or webbing, the spraying process may need to be slowed dramatically.

The surface imperfections can include strands of drug laden polymeric material hanging loosely from or extending across interstitial spaces in the medical device. These surface imperfections, because of their drug delivering capabilities, may cause adverse effects. Loose strands or strands across interstitial spaces may not be secure, and thus, may enter the blood stream and fail to provide local treatment. If these drugs are released to locations other than the targeted area, unwanted side effects may result. In addition, an uneven coating may also result in non-uniform treatment of the vessel wall.

Accordingly, there is a need for an improved method of applying a coating composition to a surface of a medical device to form a uniform coating. More particularly, there is a need for an improved method of coating a medical device by spraying a coating composition that does not drip or form webs in the interstices of the medical device. There is also a need for an efficient and cost-effective method of manufacturing such a medical device.

### SUMMARY OF THE INVENTION

These and other objectives are accomplished by the present invention. The present invention provides a method of making a coated medical device. This method comprises: (a) providing a medical device having a surface; (b) applying a coating composition to a portion of the surface; and (c) at least partially drying the coating composition applied to the surface using a heat or energy source. Step (c) is conducted substantially simultaneously with step (b) to form a coating on the surface of the medical device. Preferably, the medical device is a stent having a sidewall comprising a plurality of struts defining a plurality of openings, wherein the surface is located on the struts. The present invention also provides a coated medical device made by this method.

In another embodiment, the present invention provides a method comprising: (a) providing a stent having a sidewall comprising a plurality of struts defining a plurality of openings therein, wherein each strut has a surface; (b) applying a coating composition to at least one surface of a strut by spraying; and (c) at least partially drying the coating composition applied to the surface by applying heat or energy from a heat or energy source. Step (c) is conducted substantially simultaneously with step (b) to form a coating on the surface. Steps (b) and (c) may be repeated. The present invention also provides a coated medical device made by this method.

The present invention also provides a system for making a coated medical device. This system comprises: (a) a device for applying a coating composition to a portion of a surface of a medical device; and (b) a heat or energy source for at least partially drying the coating composition applied to the surface wherein the heat or energy source at least partially dries the coating composition substantially simultaneously with the application of the coating composition by the device.

The method and system of the present invention provide an efficient and cost-effective method of applying a coating composition to a medical device such as a stent to form a coating. By substantially simultaneously conducting the steps of (1) applying the coating composition and (2) at least partially drying the coating composition applied to the surface, the coating composition may be applied at a higher flow rate, thereby decreasing the production time. In addition, the resulting coating has reduced surface imperfections such as webbing of the coating composition between interstices on the surface of the medical device.

### BRIEF DESCRIPTION OF THE DRAWINGS

FIG. 1 shows a schematic diagram of the system and method of the present invention. In this embodiment, a nozzle apparatus sprays a coating composition onto a medical device and heat or energy is applied from a heat or energy source (not shown) to the surface of the medical device. The nozzle apparatus and heat or energy source move while the medical device remains stationary. In this figure, the heat or energy is applied inside the spray pattern.

FIG. 2 shows a nozzle apparatus and a medical device as shown in FIG. 1. The heat or energy is applied to the medical device partially inside the spray pattern.

FIG. 3 shows a nozzle apparatus and a medical device as shown in FIG. 1. The heat or energy is applied to the medical device outside the spray pattern.

FIG. 4 shows a nozzle apparatus and a medical device as shown in FIG. 1. The heat or energy is applied to the entire medical device.

FIG. 5a-f each show a nozzle apparatus and a medical device as shown in FIG. 1. In FIGS. 5a-f, the heat or energy is applied to various positions that are not centered on the medical device so that only a portion of the heat or energy from the heat or energy source (not shown) strikes the medical device.

FIG. 6 shows a nozzle apparatus and a medical device as shown in FIG. 1, with two heat or energy sources being used in conjunction with the single nozzle apparatus.

FIG. 7 shows a nozzle apparatus spraying a coating composition onto a medical device and heat or energy is applied from a heat or energy source (not shown) to the surface of the medical device inside the spray pattern. The nozzle apparatus and heat or energy source remain stationary while the medical device transverses across these devices.

FIGS. 8a and 8b show a nozzle apparatus and medical device as shown in FIG. 1 from above. In these figures, a collimated heat or energy source is applying heat or energy to the medical device. The angle between the heat or energy strikes the medical device at about a 90° angle from the spray pattern in FIG. 8a, and a less than 90° in FIG. 8b.

FIG. 9 is a schematic diagram of the system and method of the present invention. In this embodiment, a first nozzle apparatus and a second nozzle apparatus each spray a coating composition onto a medical device and two heat sources apply heat or energy from two heat or energy sources (not shown) within each spray pattern.

FIG. 10 shows a first nozzle apparatus and a second nozzle apparatus as in FIG. 9. The heat or energy is applied from one heat or energy source (not shown) to the medical device to cover both spray patterns.

### DETAILED DESCRIPTION OF THE INVENTION

The medical devices that are suitable for the present invention can be inserted into and implanted in the body of a patient. The medical devices suitable for the present invention include, but are not limited to, stents, surgical staples, catheters, such as central venous catheters and arterial catheters, guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, hemodialysis units, hemoperfusion units and plasmapheresis units.

Medical devices of the present invention include those that have a tubular or cylindrical-like portion. The tubular portion of the medical device need not be completely cylindrical. For instance, the cross-section of the tubular portion can be any shape, such as a circle, rectangle, or triangle. Such devices include, without limitation, stents and grafts. A bifurcated stent is also included among the medical devices which can be fabricated by the method of the present invention.

In addition, the tubular portion of the medical device may be a sidewall that is comprised of a plurality of struts defining a plurality of openings. The struts may be arranged in any suitable configuration. Also, the struts do not all have to have the same shape or geometric configuration. Each individual strut has a surface adapted for exposure to the body tissue of the patient. The tubular sidewall may be a stent.

Medical devices which are particularly suitable for the present invention include any kind of stent for medical purposes which is known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and Pat. No. 5,061,275 issued to Wallsten et al. Examples of appropriate balloon-expandable stents are shown in U.S. Pat. No. 5,449,373 issued to Pinchasik et al.

The medical devices suitable for the present invention may be fabricated from metallic and/or polymeric materials. Metallic material is more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646 to Mayer. Suitable metals include stainless steel, Nitinol, and Elgiloy.

Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Preferably, the medical device is pre-fabricated before application of the coatings. The pre-fabricated medical device is in its final shape. For example, if the finished medical device is a stent having an opening in its sidewall, then the opening is formed in the device before application of the coatings.

In embodiments of the present invention, the insertable or implantable portion of the medical device of the present invention has a surface. The surface may have a plurality of openings therein. Preferably, the medical device is a stent having a sidewall comprising a plurality of struts defining a plurality of openings. When the medical device is a stent comprising a plurality of struts, the surface is located on the struts.

In the present invention, a coating composition is applied to a portion of the surface of the medical device to form a coating on the surface of the medical device. Coating compositions suitable for applying to the devices of the present invention can include a polymeric material dispersed or dissolved in a solvent suitable for the medical device, which are known to the skilled artisan.

The polymeric material should be a material that is biocompatible and avoids irritation to body tissue. Preferably the polymeric materials used in the coating composition of the present invention are selected from the following: polyurethanes, silicones (e.g., polysiloxanes and substituted polysiloxanes), and polyesters. Also preferable as a polymeric material a styrene-isobutylene-copolymers. Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations of the foregoing.

More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

One or more solvents may be used with each coating composition. The solvents used to prepare coating compositions include ones which can dissolve the polymeric material into solution or suspend the polymeric material. If a biologically active material is present in the coating compositions, the solvent preferably can also dissolve or suspend the biologically active material. Any solvent which does not alter or adversely impact the therapeutic properties of the biologically active material can be employed in the method of the present invention. For example, useful solvents include tetrahydrofuran (THF), chloroform, toluene, acetone, isooctane, 1,1,1-trichloroethane, dichloromethane, and mixture thereof.

The coating composition may also include a biologically active material. The term “biologically active material” encompasses therapeutic agents, such as drugs, and also genetic materials and biological materials. The genetic materials mean DNA or RNA, including, without limitation, of DNA/RNA encoding a useful protein stated below, intended to be inserted into a human body including viral vectors and non-viral vectors. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor and platelet derived endothelial growth factor, platelet derived growth factor, tumor necrosis factor, hepatocyte growth factor and insulin like growth factor), transcription factors, proteinkinases, CD inhibitors, thyrnidine kinase, and bone morphogenic proteins (BMP's), such as BMP-2, BMP-3, BMP-4, BMP-5, BMP-6(Vgr-1), BMP-7(OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Biologically active material also includes non-genetic therapeutic agents, such as:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, and acetylsalicylic acid, amlodipine and doxazosin;
anti-inflammatory agents such as glucocorticoids, betamethasone, dexamethasone, prednisolone, corticosterone, budesonide, estrogen, sulfasalazine, and mesalamine;
antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides;
vascular cell growth promotors such as growth factors, Vascular Endothelial Growth Factors (FEGF, all types including VEGF-2), growth factor receptors, transcriptional activators, and translational promotors;
vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, biflinctional molecules consisting of an antibody and a cytotoxin;
cholesterol-lowering agents; vasodilating agents; and agents which interfere with endogenous vasoactive mechanisms;
anti-oxidants, such as probucol;
antibiotic agents, such as penicillin, cefoxitin, oxacillin, tobranycin;
angiogenic substances, such as acidic and basic fibrobrast growth factors, estrogen including estradiol (E2), estriol (E3) and 17-Beta Estradiol; and
drugs for heart failure, such as digoxin, beta-blockers, angiotensin-converting enzyme (ACE) inhibitors including captopril and enalopril.
Preferred biologically active materials include anti-proliferative drugs such as steroids, vitamins, and restenosis-inhibiting agents. Preferred restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxo triethanolamine salt, 2′-O-ester with N-(dimethylaminoethyl) glutamine, and 2′-O-ester with N-(dimethylaminoethyl) glutamide hydrochloride salt.

Other preferred biologically active materials include nitroglycerin, nitrous oxides, antiobitics, aspirins, digitalis, and glycosides as well as immunosuppressants such as rapamycin (Sirolimus).

The amount of biologically active material present in the coating composition can be adjusted to meet the needs of the patient. In general, the amount of the biologically active material used may vary depending on the application or biologically active material selected. In addition, the quantity of biologically active material used may be related to the selection of the polymer carrier. One of skill in the art would understand how to adjust the amount of a particular biologically active material to achieve the desired dosage or amount.

The polymeric material and biologically active material should be dissolved or suspended in a solvent to form a coating composition. Any suitable combination of materials may be used for the coating composition. For example, the composition may include about 90% toluene, about 5% tetrahydrofurane, and less than about 5% of the polymer and biologically active material. Preferably, the amount of the solvent is about 90% to about 99%, and more preferably about 95% to about 99%.

The coating composition can be applied by any suitable method to a surface of a medical device to form a coating. Examples of suitable methods include, but are not limited to, spraying such as by conventional nozzle or ultrasonic nozzle, dipping, rolling, and electrostatic deposition. More than one of these coating methods can be used to form the coating. A preferred method is spraying. Any spray technology may be used. For example, one suitable spraying method includes forcing the coating composition through a small orifice and atomizing the coating composition at the output by applying a compressed gas such as nitrogen. An expandable stent may be sprayed in either an expanded or unexpanded position. Preferably, a stent is sprayed in the unexpanded position.

The coating composition may be sprayed at any suitable flow rate, which can be selected by one skilled in the art. Generally, the flow rate should be slow enough to prevent the coating composition from webbing, bridging, or running down the struts of the stent. However, using the present method, the flow rates can be faster than they would be without the step of applying a heat source. Preferably, the coating composition is sprayed at a flow rate of about 20 mL/hour to about 40 mL/hour. A preferred flow rate is about 25 mL/hour.

Other spray parameters may be adjusted as known to one skilled in the art, for example. The coating composition may be sprayed in any pattern, such as in a cone pattern. In addition, the coating composition may be sprayed from any suitable device such as, but not limited to, a nozzle apparatus. The medical device may move across a nozzle apparatus as it sprays the coating composition, or the nozzle apparatus may traverse the medical device as it sprays the coating composition on the surface of the medical device.

The coating composition that is applied to a portion of the surface is at least partially dried substantially simultaneous with the application of the coating composition to the portion of the surface to form a coating on the surface of the medical device. Partially drying the composition does not include completely drying the composition, but only drying the coating composition enough to prevent running, bridging, and webbing of the coating composition between interstices on the surface of the medical device. Partially drying includes removing some, but not all, of the solvent in the coating composition. However, at least partially drying the coating composition can include completely drying the coating composition substantially simultaneous with the application of the coating composition. In addition, after the step of at least partially drying the coating composition substantially simultaneous with the application of the coating composition to the portion of the medical device, the coating composition may be completely dried or dried sufficiently to meet the specifications for residual solvents. The specific level and extent of drying can be altered at least in part in accordance with the specifications for residual solvents that are allowed to remain in the composition.

The coating composition is partially dried using a heat or energy source that applies heat or energy. The heat or energy source may apply heat or energy in any pattern, such as in a circle, ellipse, or other shape. One suitable heat energy source is a collimated or coherent heat or energy source in which the individual wavelengths are synchronized or in phase with each other and the rays remain generally parallel. Suitable collimated heat or energy sources include, but are not limited to, lasers, infrared heat sources, ultraviolet light, radio frequency energy, microwave energy, X-ray bombardment, and gamma-ray radiation. Suitable lasers including, for example, a YAG and CO₂ laser. The laser may be of any size. The infrared heat source can also be, for example, a lamp, a heater, a quartz rod, or other suitable source. The heat or energy source need not be collimated or coherent. The heat source may be a weaker heat source, such as an incoherent light such as that emitted from a light bulb.

The heat or energy source may be of any suitable wavelength as known to one skilled in the art. The collimated heat or energy source can be a wavelength to match the absorption spectrum of the solvents in the coating. The heat or energy source need not be a single wavelength.

The operating parameters of the heat or energy source depend on the stent design, spraying parameters, solvents used, and other factors as known by one skilled in the art. The heat or energy from a heat or energy source may be adjusted to obtain a desired effect. For example, a beam from a heat or energy source which has a large diameter can be run through a beam reduction optical path that will reduce the diameter and increase the energy density.

The application of the heat or energy source should not adversely affect the integrity of the materials of the medical device. Thus, the material of the medical device must by resistant to the heat or energy applied. The application of heat or energy should also not affect the integrity of any the materials in the coating composition. The biologically active material in the coating composition should not be sensitive to the wavelength of the heat or energy supplied by the heat or energy source. For example, silver nitrate will darken when exposed to light. In addition, the application of heat or energy should not cause the solvent in the coating composition to flash off or explode. The heat or energy required to partially remove the solvents should also not be greater than the energy required to break down the polymer or the biologically active material.

The heat or energy source is used to apply heat or energy to the surface of the medical device and/or to the coating composition that is applied to the surface of the medical device substantially simultaneous with the application of the coating composition to the surface. Conducting these steps substantially simultaneously means applying the coating composition and applying heat from the heat source at generally the same time. More particularly, the heat or energy is applied from the heat or energy source to the coating composition after the coating composition has been sprayed from the nozzle apparatus and before the coating composition has been completely applied to the portion of the surface. The heat or energy may be applied before the coating composition contacts the surface of the medical device, as it contacts the surface, or immediately after it contacts the surface. However, the heat or energy source should not evaporate the solvents in the coating composition before the coating composition is adhered to the surface of the medical device.

The heat or energy may be applied to the entire medical device or the portion where the coating composition is being applied. Preferably, the heat or energy is focused on the surface inside the spray pattern as the coating composition contacts the surface or just a portion of the spray pattern or any other desired position on the surface of the device. For example, as the spray is traversing along the stent or other medical device, the heat or energy source strikes the surface of the stent either inside the spray pattern or at any other desired position on the device. The heat or energy source may strike the surface at a position that trails the spray pattern or at a position that leads the spray pattern so that the device is pre-heated to achieve the drying process.

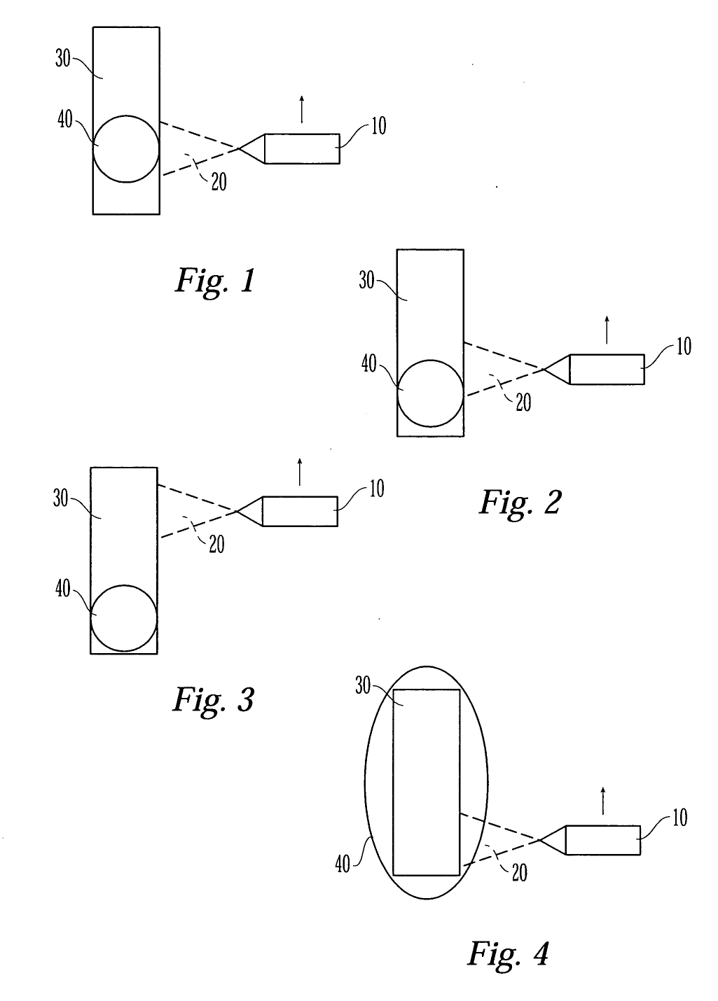

FIGS. 1-6 show schematic diagrams of the application of the coating composition and heat to a medical device 30 described above. Each of these figures shows a nozzle apparatus 10 spraying a coating composition 20 onto a medical device 30 in a cone-shaped spray pattern. These figures also show the heat or energy 40 that is applied from the heat or energy source (not shown). The heat or energy 40 is shown by a circle (FIGS. 1-3) or oval (FIG. 4). The nozzle apparatus 10 and heat or energy source move while the medical device 30 remains stationary.

More particularly, FIG. 1 shows a nozzle apparatus 10 applying a coating composition 20 in a cone-shaped spray pattern onto the surface of a medical device 30. In this figure, a heat or energy source (not shown) applies heat or energy 40 to the surface of the medical device 30 inside the spray pattern. In FIG. 2, the heat or energy 40 is applied to the surface of the medical device 30 partially inside the spray pattern. In FIG. 3, the heat or energy 40 is applied to the medical device 30 outside the spray pattern. In FIG. 4, the heat or energy 40 is applied to the entire medical device 30.

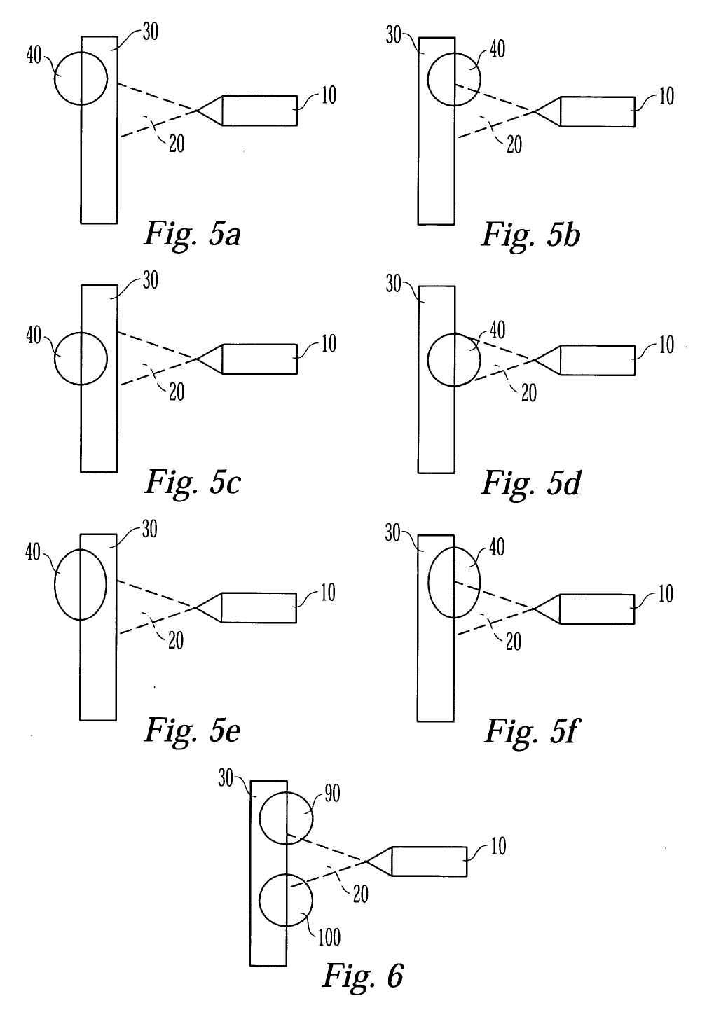

The heat or energy source can be applied to any part of the medical device 30. For example, FIGS. 5a-f show a nozzle apparatus 10 and a medical device 30 as shown in FIG. 1. In FIGS. 5a-f, the heat or energy 40 is applied to various positions that are not centered on the medical device 30 so that only a portion of the heat or energy 40 from the heat or energy source (not shown) strikes the medical device 30.

More than one heat or energy source may be used to apply the heat or energy. FIG. 6 shows a nozzle apparatus 10 and a medical device 30 as shown in FIG. 1 in which two heat or energy sources are used in conjunction with the single nozzle apparatus 10. The heat or energy sources apply heat or energy 90, 100 to the medical device 30.

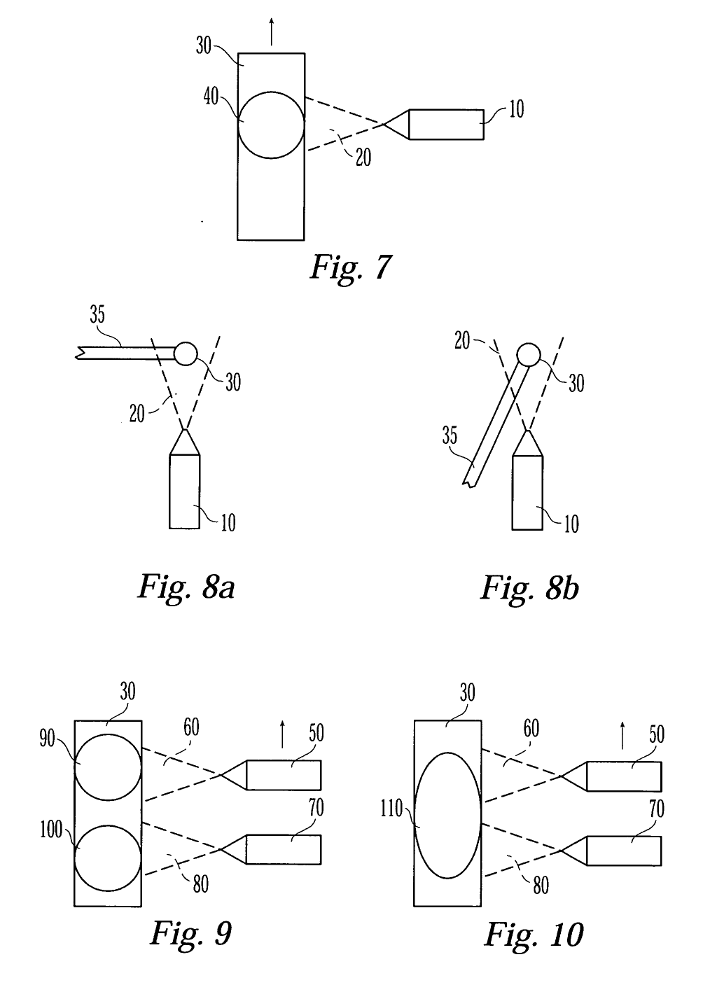

In addition, the device for applying the coating composition 20 and the heat or energy source may move or the medical device 30 may move as the nozzle apparatus 10 moves along the medical device 30. The heat or energy source preferably follows and maintains the same position with respect to the spray pattern. FIG. 7 shows a nozzle apparatus 10 spraying a coating composition 20 onto a medical device 30 and a heat or energy source (not shown) applying heat or energy 40 to the surface of the medical device 30 inside the spray pattern. In FIG. 7, the nozzle apparatus 10 and heat or energy source remain stationary while the medical device 30 moves across these devices.

The heat or energy may be applied to the surface of the medical device 30 from any suitable angle with respect to the medical device 30 and spray pattern. In FIGS. 1-7, the heat or energy source is applied from an angle of approximately 90° from spray pattern. FIGS. 8a and 8b show a nozzle apparatus 10 and medical device 30 as shown in FIG. 1 from above. In these figures, a collimated heat or energy source (not shown) is applying heat or energy 35 to the medical device 30. The angle between the heat or energy strikes the medical device 30 at about a 90° angle from the spray pattern of the coating composition 20 in FIG. 8a, and at less than about 90° in FIG. 8b.

The process of applying the coating composition 20 to the surface substantially simultaneous with the application of heat or energy 40 from a heat or energy source to at least partially dry the coating composition 20, may be repeated one or more times to form a coating on the surface of the medical device 30. In other words, the coating composition 20 may be applied in one or more passes. The process may be repeated until a desired amount of the coating composition 20 has been applied. In addition, the process may be repeated using different coating compositions 20. The coating may be formed by a single pass or multiple passes of the spray pattern to form the coating on the medical device 30. Preferably, the coating layer is formed in a single pass. By partially drying the coating composition 20 before applying the next pass of the coating composition 20, there are not discrete multiple layers. Instead, this method results in a single coating on the surface of the medical device 30.

In another embodiment, after the step of at least partially drying the coating composition 20 simultaneous with the application of the coating composition 20, the coating composition 20 may be further dried to remove most or all of the solvents. The coating composition 20 may be further dried after each pass or only after the last pass of the coating composition 20.

In addition, the process may be repeated using different coating compositions. A first nozzle apparatus 50 may spray a first coating composition 60 and a second nozzle apparatus 70 may spray a second coating composition 80. In addition, one or two heat or energy sources may be used to apply heat or energy 90, 100, 110 to the surface of the medical device 30 substantially simultaneous with the application of the coating compositions. For example, FIGS. 9 and 10 show a first nozzle apparatus 50 that sprays a first coating composition 60 and a second nozzle apparatus 70 that sprays a second coating composition 80 onto a medical device 30. In FIG. 9, the heat or energy 90, 100 is applied from two heat or energy sources (not shown) inside each spray pattern of the coating composition 60. In FIG. 10, heat or energy 110 is applied from one heat or energy source (not shown) to the medical device 30 to cover the spray patterns of the first coating composition 60 and second coating composition 80.

The system of the present invention includes a device for applying the coating composition to a portion of a surface of a medical device, and a heat or energy source for at least partially drying the coating composition applied to the surface. As explained above, the heat source at least partially dries the coating composition substantially simultaneous with the application of the coating composition by the device. Suitable devices and heat or energy sources include those described above.

In use, a coated medical device, such as an expandable stent, of the present invention may be used for any appropriate medical procedure. The coating medical device is inserted into a body lumen where it is positioned to a target location. Delivery of the medical device to a body lumen of a patient can be accomplished using methods well known to those skilled in the art, such as mounting the stent on an inflatable balloon disposed at the distal end of a delivery catheter. The biologically active material diffuses through the coating to the body lumen. This enables administration of the biologically active material to be site-specific, limiting the exposure of the rest of the body to the biologically active material.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

1. A method of making a coated medical device comprising:
(a) providing a medical device having a surface;
(b) applying a coating composition to a portion of the surface; and
(c) at least partially drying the coating composition applied to the surface using a heat or energy source, wherein step (c) is conducted substantially simultaneously with step (b) to form a coating on the surface of the medical device.

2. The method of claim 1, wherein the surface of the medical device has a plurality of openings therein.

3. The method of claim 1, wherein the medical device is a stent having a sidewall comprising a plurality of struts defining a plurality of openings, and the surface is located on at least one strut.

4. The method of claim 1, wherein the coating composition comprises a polymer.

5. The method of claim 1, wherein the coating composition comprises a biologically active material.

6. The method of claim 5, wherein the biologically active material comprises at least paclitaxel or rapamycin.

7. The method of claim 1, wherein the coating composition is applied by spraying.

8. The method of claim 7, wherein the coating composition is sprayed at a flow rate of about 10 mL/hour to about 40 mL/hour.

9. The method of claim 1, wherein the heat source is a collimated heat source.

10. The method of claim 1, wherein the heat source is a non-collimated heat source.

11. The method of claim 1, wherein the energy source is a collimated energy source.

12. The method of claim 1, wherein the energy source is a non-collimated energy source.

13. The method of claim 9, wherein the collimated heat source is a laser, an infrared heat source, radio frequency radiation, microwave radiation, X-ray radiation, or gamma-ray radiation.

14. The method of claim 11, wherein the collimated energy source is a laser, an infrared heat source, radio frequency radiation, microwave radiation, X-ray radiation, or gamma-ray radiation.

15. A coated medical device made by the method of claim 1.

16. A method of making a coated stent comprising:
(a) providing a stent having a sidewall comprising a plurality of struts defining a plurality of openings therein, wherein each strut has a surface;
(b) applying a coating composition to at least one surface of a strut by spraying; and
(c) at least partially drying the coating composition applied to the surface by applying heat or energy from a heat or energy source, wherein step (c) is conducted substantially simultaneously with step (b) to form a coating on the surface.

17. The method of claim 16, wherein the coating composition comprises a polymer.

18. The method of claim 18, wherein the coating composition comprises a biologically active material.

19. The method of claim 16, wherein the biologically active material comprises paclitaxel or rapamycin.

20. The method of claim 16, wherein the coating composition is sprayed from a nozzle apparatus onto the surface at a flow rate of about 10 mL/hour to about 40 mL/hour.

21. The method of claim 16, wherein the heat or energy source is a laser.

22. A coated medical device made by the method of claim 16.

23. A system for making a coated medical device comprising:
(a) a device for applying a coating composition to a portion of a surface of the medical device; and
(b) a heat or energy source for at least partially drying the coating composition applied to the surface wherein the heat or energy source at least partially dries the coating composition substantially simultaneously with the application of the coating composition by the device.

24. The system of claim 23, wherein the device applies the coating composition by spraying.

25. The system of claim 24, wherein the device is a nozzle apparatus.

26. The system of claim 25, wherein the nozzle apparatus sprays the coating composition at a flow rate of about 10 mL/hour to about 40 mL/hour.

27. The system of claim 25, wherein the longitudinal axis of the nozzle is substantially perpendicular to the longitudinal axis of the medical device.

28. The system of claim 25, wherein the longitudinal axis of the nozzle is substantially non-perpendicular to the longitudinal axis of the medical device.

29. The system of claim 25, wherein the heat or energy source is a collimated heat or energy source.

30. The system of claim 29, wherein the collimated heat or energy source is a laser, an infrared heat source, radio frequency radiation, microwave radiation, X-ray radiation, or gamma-ray radiation.

31. The system of claim 25, wherein the surface of the medical device has a plurality of openings therein.

32. The system of claim 25, wherein the medical device is a stent having a sidewall comprising a plurality of struts defining a plurality of openings, and the surface is located on at least one strut.
